(19) United States
(12) Patent Application Publication
(10) Pub. No.: US 2016/0206768 A1
(43) Pub. Date: Jul. 21, 2016

(54) METHOD AND SYSTEM FOR PRODUCING, STORING, AND DISPENSING OF IODINE, VITAMIN AND MINERAL ENRICHED WATER

(71) Applicant: NEET INNOVATIONS, LLC, Mount Airy, GA (US)

(72) Inventor: Terence F. MULLINS, Quebec (CA)

(21) Appl. No.: 15/024,090

(22) PCT Filed: Sep. 23, 2014
(86) PCT No.: PCT/US2014/056895

§ 371 Date: Mar. 23, 2016

Related U.S. Application Data
(60) Provisional application No. 61/881,052, filed on Sep. 23, 2013.

(51) Int. Cl. A61L 2/22; A61L 9/14; A01N 59/12

(52) U.S. Cl. CPC A61L 2/22; A01N 59/12; A61L 9/145

(57) ABSTRACT

A method and apparatus that produces formulated iodine enriched water for use with multiple applications. The iodine enriched water is transformed through humidification processes within a mobile device producing iodine enriched air within a room or establishment. A portable iodine container can be used to receive the iodine enriched water for use in disinfection applications to the atmosphere and surfaces and in nebulizing applications. The iodine enriched water can include vitamins and/or minerals, if desired.

This application claims priority under 35 USC 119(e) based on application No. 61/881,052, filed on Sep. 23, 2013, which is herein incorporated by reference in its entirety.

### FIELD OF THE INVENTION

The present invention relates to methods and mobile portable devices to produce formulated iodine enriched water, retain it, and modify it. Uses include but are not limited to human health applications, topical disinfection uses, surface and air disinfection applications and human drinking water applications.

### BACKGROUND ART

Iodine is used is currently used as a disinfectant in hospitals. Iodine is well recognized as a world class disinfectant used against bacteria and viruses which constitute a major health risk to humans. Medical facilities due to their nature often propagate the proliferation of disease among both healthy and sick individuals.

The vectors for the transmission of disease within medical establishments are mainly airborne and surface borne. Post-operative infections are a common occurrence that cause pain and undue suffering for humans and create a cost increase in patient care and treatment in the 40 billion dollar range annually.

Current air scrubber systems and air filtration system are not adequately controlling the microbiological loading being created with a given medical environment. To date, no alternative has been found to address this growing concern. Currently 1.7 million acquire disease while visiting a hospital in the US and 100,000 people die from diseases acquired from the hospital.

Iodine is a required and an essential human and animal micronutrient. Methods of delivering iodine into our bodies have been by either ingestion or intravenous. There is a growing body of scientific work determining the effects of inhalation therapy as a more effective delivery means for certain treatments. Due to the absence of a safe pure iodine inhalation system, to date there have been no inhalation health studies with iodine. It has been proven that certain vitamins and minerals act as auto-catalysts to facilitate the adsorption of certain essential micronutrients within the human body.

The production of aqueous iodine solutions is well understood. However, under dynamic flow conditions, current technologies fail due changing water temperatures within the iodine bed and fluctuating dissolution rates caused by water channeling issues within the iodine bed.

Heating the primary water supply was identified as a stabilizer in the dissolving of iodine crystals under dynamic flow conditions. The problems with this design occur not during operation but when the system is dormant and between operations. Exterior environmental temperatures change the iodine solution temperature and thereby the concentration within the cylinder. Systems fail because they does not recognize this problem and only rely on temperature to determine the iodine concentration. For example, if a known concentration is produced based on a given temperature and then the iodine bed sits dormant, the iodine concentration will change based on ambient or other sources of heat. If the temperature is then reduced in said iodine bed, the concentration will remain at its most concentrated level. So, temperature cannot be used as an indicator of iodine concentration within a solution or an iodine bed.

Iodophor/Povodone are the current iodine technologies used in hospitals. The only active biocide is iodine in these technologies. By design, their molecular structures utilize material within the matrix's that are not suitable for human consumption or inhalation. Plastics, surfactants, and acids are among the inert materials.

It is also well understood that at low pH levels below 8, elemental iodine is the dominant species of iodine and is a superior biocide. Hypoiodous acid, which is present at high pH levels above 8, is the dominant species of iodine and a superior virucide. However, delivering these products through a fine or coarse mist spray does not affect the disinfection capabilities of iodine.

Iodine to date has not been utilized as a disinfectant through central or portable humidification/spray systems due to several factors including open reservoir water supply, surface retentions and contact time of the disinfectant, premature release of the molecular iodine bonding with the carrier molecule, location and design of the humidification spray system and the inability to modify the drop size of the water molecule. Also, the inability to accurately deliver a known concentration of iodine therefore producing a known iodine disinfectant concentration is not possible.

There is currently no technology that produces iodine, vitamin and mineral enriched water for human consumption. There is currently no system that produces iodine enriched air for any use.

The present invention addresses and surmounts the hereinabove debilitating factors for humidification/spray applications employing aqueous iodine as a disinfectant and iodine being accompanied by mineral and vitamin supplements.

### SUMMARY OF THE INVENTION

Iodine enriched water produces iodine enriched air when delivered through a humidifier system within a room or establishment. Iodine or a combination of iodine, vitamins and minerals are infused into air in their aqueous states by water droplets produced through humidification processes within a system to provide for aerosol disinfection, surface disinfection, or human health inhalation therapy. Iodine is the only biocidal element infused into the air by the system. The concentration of iodine produced within a cubic meter of air is controlled through a combination of humidification processes and the quantity of iodine within the solution to be humidified. The present invention includes mobile humidification spray devices employing fine dispersion mists by way of humidification processes and a coarse dispersion trigger spray system to provide wetted contact surface disinfection or topical disinfection. Thermodynamic systems manipulate the disinfectant temperature and thereby control the retention of said iodine within the H₂O bond in the humidification process. The invention provides for iodine enriched humidified air containing variable water droplet sizes from small to large to accommodate human inhalation particle size indices, aerosol disinfection, and surface disinfection droplet particle sizes. The percentage of droplet sizes can be modulated and regulated within the humidified air produced by the device by adjusting the ratio mixture between the ultrasonic and impeller humidifier systems. A fine water mist produced by a standard off the shelf ultrasonic humidifier within the device more readily and evenly disseminates the aqueous iodine within a volume of air. The small water droplet particle size produced by the ultrasonic humidifier system may be carried evenly throughout a room or establishment and may also be inhaled deeply into human lungs for therapeutic and micronutrient uptake health effects. A large particle mist produced by a standard off the shelf impeller humidifier within the device produces humidified air with larger heavier water droplets thereby producing heavier more dense air that will coat surfaces within a room and provide for contact surface disinfection to control air borne and surface borne microbes in medical, industrial, commercial, agriculture and home environments. The combination of light humidified iodine enriched air and heavy humidified iodine enriched air within a room or establishment will attain superior microbial control in the air and on surfaces. The present invention includes a mobile device with telescopic venting ports to infuse iodine enriched humidified air at variable heights and locations within a room or establishment. Lighter iodine enriched air will reach the upper recesses within a room providing disinfection in areas that are normally difficult to control. Heavier iodine enriched air that is generated will sink vertically over time to kill surface borne microbes.

As a method of superior surface disinfection or topical wound disinfection, a hand held coarse trigger spray system with a flexible extendible tube apparatus on the device delivers aqueous iodine in a coarse spray. Used as a hard surface direct contact disinfectant to kill surface borne microbes within a room where the device is utilized or sprayed on a patient's wounds. The coarse trigger spray system draws its iodine supply from a distinct iodine canister within the mobile device.

The present invention includes a device with a human nebulizer apparatus to enable patients or people to breathe higher concentrations of iodine, vitamin and mineral enriched air directly from the device by way of a nebulizer face mask attached to the mobile humidification spray device. The nebulizer draws its iodine enriched air through a distinct ultra sonic humidifier within the device. The ultrasonic humidifier draws its iodine, vitamin and mineral and or vaccine supply from distinct canisters within the mobile device.

The present invention provides for iodine, vitamin, and mineral tanks that are refillable and are mated to both the iodine dispensing tanks at the portable filling station and the mobile humidification spray devices.

The present invention includes a portable iodine filling station utilizing a method of producing a known concentrate of iodine enriched water, retaining and dispensing said concentrate, reducing said concentrate and independently manipulating the iodine species.

The filling station produces high quality water that is then infused with a formulated iodine, and or vitamins and mineral concentrates. Blend ratio formulas of the concentrates are factory preprogrammed into an electronic controller to produce iodine and vitamin and mineral enriched water at specific levels depending on the required use of the end product. The iodine, and or vitamin and mineral enriched water may be consumed for health or utilized to produce iodine enriched air when humidified. Formulas also include topical wound disinfection applications including but not limited to minor cuts and abrasions or post surgery site disinfection. For example, high quality water is infused with formulae of iodine, vitamin E and or vitamin C.

The present invention includes devices on the filling station to monitor and control the production of the aqueous iodine solutions, an apparatus to retain, filter and manipulate the water chemistry of a given water supply called the high quality blend water. The high quality filtered blend water is infused with the aqueous iodine concentration to produce controlled iodine enriched water. The concentration of iodine enriched water determines whether it can be consumed as drinking water or inhaled as a human micronutrient supplement, or an agent to reduce or destroy disease causing organisms within the human body or as a broad band microbial surface and aerosol iodine disinfectant. The iodine enriched water is stored in iodine dispensing tanks within the portable iodine filling station.

One object of the invention is to produce a concentrate of high quality iodine enriched water by infusing high quality water with a concentrated aqueous iodine to produce different concentrations of iodine enriched water that may or may not be infused with vitamins and minerals. The iodine enriched water is then used to produce iodine enriched air by way of humidification processes, which when put within a room, kills airborne microbes. Iodine enriched air also kills microbes on surfaces that the air within a room comes in contact with. Concentrations levels of iodine enriched air within a room that are fatal to microbes are safe and healthy for humans breathe. Another object of the invention is to provide a mobile apparatus or station that can be moved from room to room within an establishment and deliver iodine enriched humidified air to ensure that the room is safe from airborne and surface microbes.

A further object of the invention is to provide an apparatus and method for producing and retaining iodine enriched water that dispense the dispensing iodine enriched water through a refillable, mobile, variable humidification spray system creating iodine enriched air to provide surface and air microbial control within a room or establishment. The iodine species can be manipulated through pH control within the iodine dispensing tanks to target specific airborne and surface borne bacteria and viruses. The invention provides for the modulation and blending of iodine species to accomplish the aforementioned target specific speciation. The invention provides for the control of the retention bond between the iodine species and the carrier water molecule through a cooling process of the iodine solution within the device as well as for altering the dimensions of the water drop in the humidification or spray produced to cover the full range of water droplet sizes that can be produced with conventional humidification processes. The invention also provides for inhalation therapy with iodine, vitamins, minerals, and vaccines with a nebulizer attachment on the mobile device.

There are three primary aspects of the overall invention, an iodine filling station, a mobile chemical spray device, and one or more refillable iodine canisters.

The portable iodine filling station produces and dispenses controlled species of iodine enriched water at a remote location such as a hospital or clinic.

The invention also provides a plurality of dispensing tanks for the variable iodine solutions being retained.

One other object of the invention is to have a bladder within the dispensing tanks made of iodine inert material such as teflon to retain and maintain the integrity of the aqueous iodine solutions. The distinct dispensing tanks can be mated with dispensing fittings between the tanks and the distinct iodine concentration specific canisters.

Yet another object of the invention is to produce controlled levels of iodine enriched air within a room or establishment and to produce vitamin and mineral enriched air within a room or establishment.

Another aspect of the invention includes a nebulizer system for direct human inhalation of the iodine and or vitamin, mineral enriched air.

The invention also provides for a multi functional, mobile chemical spray device that provides a coarse spray device to provide microbial control on hard surfaces. The multi functional, mobile chemical spray device provides a coarse spray that can be used as a topical disinfectant for cuts and abrasions and post surgery sites.

A further object of the invention is to thermodynamically control the iodine solutions within the mobile chemical spray system. The multi functional, mobile chemical spray device is portable and may easily be transported between rooms within an establishment. The mobile spray device can include flexible telescopic vents to directionally deliver the iodine enriched humidified air at different points within a room or establishment and/or control and vary the water particle size produced in the iodine enriched humidified air. The mobile chemical spray device can also control and vary the percent of iodine enriched humidified air being produced. The iodine, mineral and vitamin solutions concentrations being delivered through an electronic controller can be controlled to vary the iodine, mineral, and/or vitamin concentrations.

Another object of the invention is to provide refillable iodine solution canisters within the portable mobile chemical spray device and to provide distinct canisters with iodine specific concentrations. The iodine canister can include a chemical inert collapsible bladder within the iodine canister and canisters with microbial species targeted iodine solutions. The distinct iodine canister connections can be sized accordingly to specific iodine disinfectant concentrations being contained within the canister.

The multi functional, mobile chemical spray device can have specific size connection receptors based on refill iodine dispensing tank to iodine canister and iodine canister to mobile chemical spray device.

In another embodiment of the invention, there are no bladders within the iodine dispensing tanks and the iodine canisters. In this embodiment, the canisters and the tanks are a fabricated spun fiberglass construct with carbon-filtered pressure relief valves. In this embodiment, small quantities iodine will be released from the solution into the air within the tank, hence the reason for the carbon filter on the pressure relief valve.

The iodine canisters can be filled with filtered water and the aqueous iodine solution can be injected by a portable aqueous iodine tube and roller delivery system. In this embodiment, aqueous iodine is dispensed from a chemically inert liquid retaining bag or container.

The primary aqueous iodine concentration tank in the filling station is filled by a portable iodine generator. Telescopic tubular air vents can be provided that oscillate to enhance delivery of the iodine-enriched air. The iodine canisters can be filled directly from the iodine concentration tank and blend water tank. In this embodiment, any alteration of the iodine species can occur at the fill point.

In another embodiment of the invention, iodine, vitamins and minerals are delivered through a nebulizer system to combat human health issues within the body including micronutrient support of the immune system and combating cancer cells. Preferably the filling station apparatus described above can further comprise reverse osmosis filtration of the incoming water source, wherein water pressure moves the water through the filter into a holding tank. From the holding tank, peristaltic or positive displacement pumps can move the water supply from the holding tank to the iodine generator and then onto the iodine concentration tank. Concurrently the holding tank also provides pressurized water to the plurality of iodine tanks. Peristaltic pumps move the iodine from the concentration tank to be infused into the blend water from the holding tank to fill the iodine disinfectant tanks. Electronic valves on the iodine tanks allow for filling of the tanks. Peristaltic pumps or other controls can be employed to control the pH of the iodine disinfectant. An electronic programmable controller receives inputs to manage blending the iodine and logging the gallons of aqueous iodine produced and dispensed. The electronic controller will cease operation, i.e., engage a safe mode if out of range inputs are detected that cannot be rectified.

Preferably the mobile chemical spray apparatus described above can further comprise multiple known humidification technologies, including ultrasonic and impeller types, and an electronic controller to adjust and set the desired humidification levels. Telescopic tubular vents that are air flow adjustable, flexible, and fabricated from iodine inert materials can be provided. Multiple fans can be used to control the airflow volume through the telescopic tubular vents. A self priming hand trigger spray apparatus with an extendable source tube can also be provided as well as a nebulizer attachment that integrates directly into the humidification system. A cooling system that controls the temperature of the iodine solutions being humidified can also be employed. The filling station apparatus can preferably include the following components to produce the desired iodine enriched water for use as desired:
a) a flow sensor to detect and an electronic valve to control the source water flow;
b) a reverse osmosis water filter;
c) a temperature controlled pressure tank to retain the filtered water;
d) an aqueous iodine generator with refillable recharges;
e) an iodine analyzer to detect the iodine concentration in the retaining tank;
f) a pump to blend the iodine concentrate into the blend water filling the iodine disinfection tanks;
g) a pump to adjust the pH of the iodine solutions;
h) a pump to add soluble minerals and vitamins;
i) refillable vitamin and mineral canisters;
j) a plumbing connection between the iodine concentration tank and the iodine disinfectant tanks and the filtered water tank;
k) mated filling connections between the iodine tanks and the iodine canisters;
l) a programmable controller that collects data on the operation of the system;
m) a programmable controller that can access data remotely produced by the system;
n) a programmable controller with safety limits built in;
o) a programmable controller with interactive displays; and
p) a mobile portable enclosure that houses all the filling station components.

The mobile chemical spray device can preferably include the following components to produce iodine, vitamin and mineral enriched air; and provide for surface and air disinfection. A nebulizer provides for human inhalation therapy:

A) an ultrasonic humidifier;

B) an impeller humidifier;

C) telescopic tubular vents;

D) multiple fans to control the output of the humidification system;

E) a nebulizer;

F) refillable aqueous iodine canisters;

G) refillable vitamin canisters;

H) refillable mineral canisters;

I) a coarse trigger spray apparatus;

J) a programmable controller that can provide intermittent programming;

K) a programmable controller with interactive displays;

L) a programmable controller with safety limits built in;

M) a programmable controller with interactive displays; and

N) a mobile portable enclosure that houses all the chemical spray device components.

The present invention targets and addresses the current problems with microbiological control within hospitals and medical establishments. The invention provides the broadest spectrum of iodine microbial control within the general and aerosol disinfectant marketplace. This invention provides aerosol and surface disinfection that can be applied on an intermittent basis to kill airborne diseases and a surface disinfectant to manage or eradicate harmful pathogens specifically bacteria and viruses. The present invention also provides a nebulizer for inhalation therapy.

The present invention provides for a myriad of iodine species and formulas accompanying iodine with application specific minerals and vitamins. Iodine enriched air produced by humidified iodine enriched water with iodine solution levels ranges from 0.25 ppm to 600 ppm aqueous iodine.

Accordingly, in further aspect of the invention provides iodine species that are contained in an aqueous state prepared and formulated according to the invention as hereinabove defined for use in the aforesaid applications.

### BRIEF DESCRIPTION OF DRAWINGS

In order to better understand the invention, the preferred embodiments will now be described by way of example with references to the drawings.

FIG. 1 illustrates a schematic flow diagram of one embodiment of a method and device mobile portable iodine filling station apparatus according to the invention.

FIG. 2 illustrates a schematic flow diagram showing alternative methods and apparatus according to the filling station invention.

FIG. 3 illustrates a schematic flow diagram of one embodiment of the chemical spray apparatus according to the invention.

FIG. 4 illustrates a schematic flow diagram of an alternative apparatus to that shown in FIG. 3.

FIG. 5 illustrates a diagram of one embodiment of the iodine canister according to the invention.

### DETAILED DESCRIPTION OF THE PREFERRED EMBODIMENTS

The chemical delivery system of the invention provides for multiple aqueous iodine canisters. for medical applications, comprising dissolving solid iodine into a first filtered water flow thereby producing a concentrated aqueous iodine solution; storing the iodine solution and then reconstituting the concentrated iodine solution by blending it into a second filtered water flow to produce pre-determined iodine disinfectant concentrations of aqueous iodine.

The iodine filling station is an apparatus that is an interconnected series of tanks to retain, mix, and dispense upon demand aqueous iodine that is chemically adjusted to polarize the species of iodine.

Portable canisters are filled with aqueous iodine disinfectant from one of the filling stations' iodine disinfectant tanks by way of a mated dispensing system. The iodine disinfectant canisters are then inserted into a portable, mobile, coarse (spray trigger) and fine spray (impeller or ultrasonic humidifier) chemical delivery system with mated connections to ensure disinfection concentration and application specificity safety. The iodine disinfectant concentration is then thermodynamically adjusted within the chemical delivery system to maximize retention and disinfection variables. The chemical delivery system can then be transported to areas where surface or air disinfection is required, typically a hospital room or critical care room.

The invention provides for a chemical delivery device that is multi-functional, portable, and mobile and provides for a coarse spray surface disinfectant and fine aqueous iodine aerosol spray disinfectant. In the present invention in one mode, the chemical delivery device can be in the shape of a vertical tower that is rectangular and mounted on wheels for mobility. The tower comprises a mounting and receptacle configuration to receive and retain the iodine disinfectant canisters. The fine spray is generated by an impeller and/or ultrasonic humidification apparatus within the tower and wetted parts are modified to be chemically inert to iodine. The humidification apparatus utilizes the iodine canisters as the source liquid to produce iodine enriched humidified air. The tower can have multiple flexible tubular telescopic air vents emanating from the humidification apparatus. The telescopic vents provide maximum air exposure and coverage for the disinfectant within a contained environment. The tower also provides controls to increase or reduce the humidity level and water drop size delivered within the air of an environment, thereby controlling the iodine level within a cubic meter or air; water drop size also determines the degree of inhalation within the pulmonary system. The tower also can thermodynamically control the temperature of the iodine canisters thereby controlling the bond between the iodine and the carrier molecule. Controlling the molecular bond provides an iodine time release method to produce both surface and aerosol disinfection.

The tower can also provide for specific surface disinfection by way of a hand held adjustable coarse spray trigger pump. The trigger pump is fed disinfectant by a designated iodine disinfectant canister within the tower. The trigger pump is connected to the tower by way of an extendable hose thereby providing critical point surface disinfection at all locations within a room.

The iodine canisters in the present invention can be rectangular. The canisters can retain approximately 6 liters of aqueous iodine disinfectant. The tanks are fabricated from chemically inert materials such as teflon or the like. The iodine canisters are sealed and have specific nozzle filling and dispensing connections. The canister connections are size mated based on the product label on the canister's designating concentration and use. The canisters are designed to be chemical free handling and fit accordingly into the chemical delivery system.

The invention provides variable disinfection concentrations and microbiological species targeting through a programmable controller and a method for producing target specific iodine species to harness and maximize the disinfection capabilities of iodine, which includes modulation capabilities of the iodine species and iodine concentration.

The invention targets and addresses the current problems with microbiological control within hospitals and medical establishments. The invention provides the broadest spectrum of iodine microbial control within the general disinfectant marketplace. This invention safely provides both and aerosol disinfectant to kill airborne disease and a surface disinfectant that can be used on a continuous or intermittent basis to manage or eradicate harmful pathogens specifically bacteria and viruses. The present invention provides for a myriad of iodine species configurations, retention times within the iodinated mist water and disinfection treatment levels ranging from 0.25 ppm to 300 or even up to 600 ppm aqueous iodine. The invention also can provide controlled aqueous iodine solutions for the ocean industries to control zebra mussel's migration and other ballast water microbes, micronutrient applications for humans, animals and plants, inhalation of iodine for the reduction of tumors in humans, and treatment for iodine deficient humans

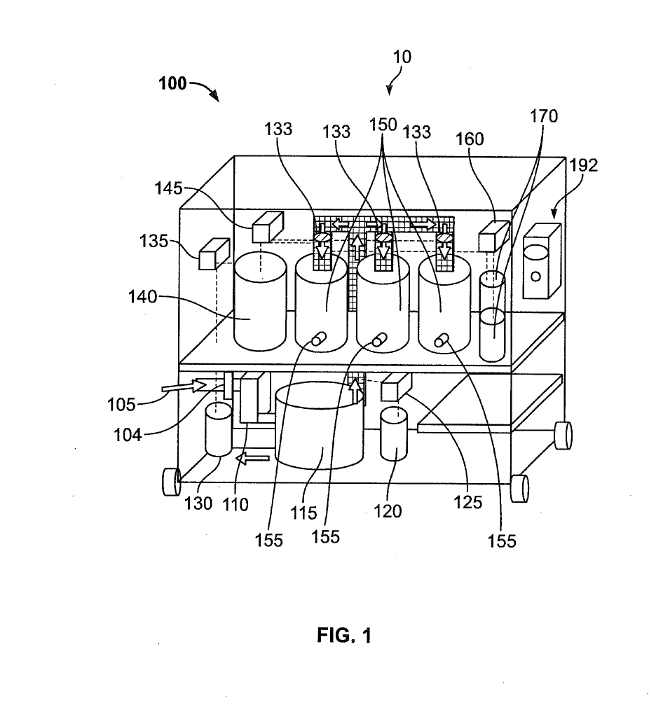

Referring now to the drawings and particularly FIG. 1, FIG. 1 shows a mobile dispensing station 10. The dispensing station 10 includes a series of chemical retaining tanks with the accompaniment of a 99.8% solid iodine based aqueous iodine generator. An incoming potable water source is introduced to the system at inlet water pipe 105. The source water is controlled by electronic valve 104 and can pass through a reverse osmosis filter 110 or other filter if filtering is desired or needed (dependent on the source of water). The filtered water is then retained in a plastic pressurized tank 115. The water tank 115 supplies water for two different functions. One is the water supply to an aqueous iodine concentrate generator 130. This iodine generator is referenced in patent application Ser. No. 14/205,690, which is incorporated herein in its entirety by reference. The iodine concentrate from the generator 130 is then pumped by peristaltic pump 135 into iodine concentrate retention tank 140.

The iodine concentrate is then pumped by peristaltic pump 145 in iodine disinfectant tanks 150. A controller 192 determines the quantity of iodine concentration delivered to each tank 150. An electronic valve 133 is associated with each tank 150. Each valve 133 opens and allows water from water tank 115 to fill respective tanks 150. The controller 192 receives sensory inputs to determine liquid levels within each tank. Minerals and vitamins are retained in tanks 170 and pumped by peristaltic pump 160 into tanks 150. The controller 192 determines the addition of the vitamins and minerals.

The iodine disinfectant is then are dispensed from tanks 150 by way of concentration specific dispensing connections 155, which means that each connection 155 is sized to mate with a complementary connection on an iodine canister, also associated with a given concentration. For example, one tank may have iodine at 25 ppm concentration and the connection at the tank is designed to interface only with another canister or the like designed to also have 25 ppm concentration for a desired use.

The pH of the water in tank 115 is controlled by way of chemicals in tank 120. The pH adjusting chemical is pumped by peristaltic pump 125 into the water line exiting tank 115, which subsequently is used to fill tanks 150.

In use of the station 10, water enters the system, is filtered, and retained in holding tank. Water from the holding tank then passes through an iodine generator and a concentrated aqueous solution is produced and stored in a holding tank.

Water from the holding tank is then blended with the iodine concentrate and or minerals and vitamins, the blend then stored in distinct holding tanks. These holding are ready to be used for dispensing into small portable canisters that will contain a predetermined concentration of iodine and, optionally, vitamins and minerals.

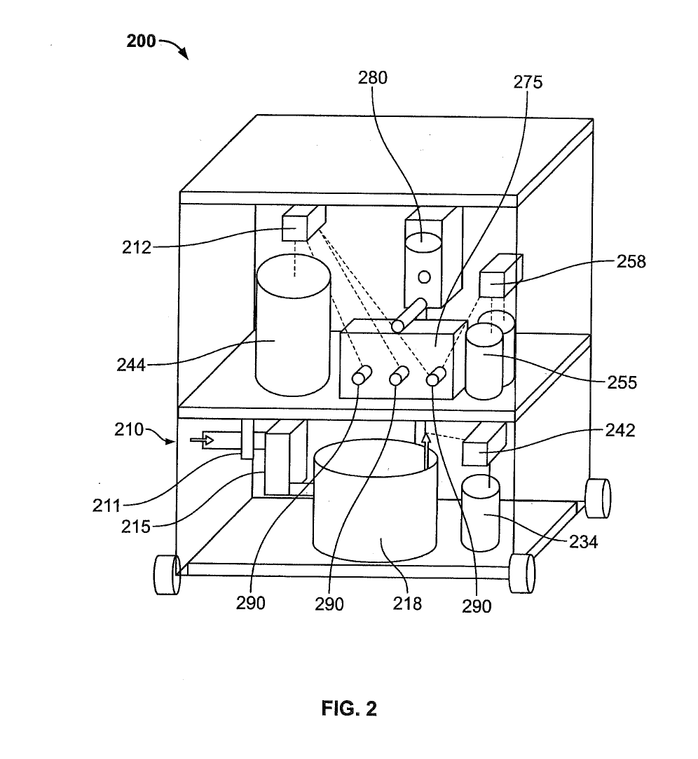

FIG. 2 is a variation on the dispensing station 10 of FIG. 1. In FIG. 2, there are no holding tanks. Instead, a water manifold is used to dispense the desired iodine enriched water for subsequent use. Referring to FIG. 2, potable water enters through inlet pipe 210 and its flow is controlled by electronic valve 211. The water then then passes through reverse osmosis filter 215 or other filter if necessary. The water then enters a plastic pressurized tank 218. pH adjustments to the water are made by the pH adjustment chemical stored in container 234 and pumped by peristaltic pump 242 to the tank 218. Water then flows to the water manifold 275 and is split into multiple streams and dispensed at spigots 290. The 290 spigots have distinct size specific mated fittings like those described in FIG. 1.

The concentrated aqueous iodine is retained in tank 244 and the iodine is proportionally metered with peristaltic pump 212 to injection points just prior to spigots 290. Minerals and vitamin via tank 255 can be proportionally injected by pump 258 just prior to spigots 290 as well. An electronic controller 280 is provided that is a failsafe programmable controller that receives input signals from the system to accurately blend the iodine concentrate, water, and the minerals and vitamins to dispense a plurality of aqueous iodine species and iodine, mineral, and vitamin blends at the disinfectant dispensing point, i.e., spigots 290.

The pumps and mixing manifold 275 is used to combine the concentrated iodine solution with the filtered water and the optional vitamins and minerals are considered the means for mixing these components together to produce the iodine enriched water of desired concentration for supply to the spigots 290 and further use in a particular application for storage for use later.

Again, the FIG. 2 embodiment provides the desired blend of concentrated iodine, water, vitamins, and minerals in an on-the-fly approach that does not require the storage of the blend as is done in FIG. 1.

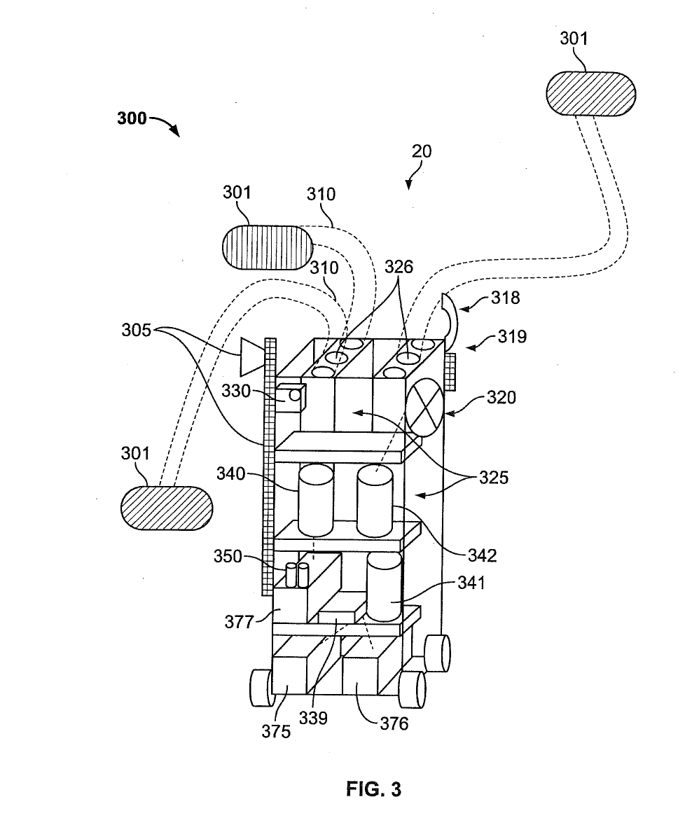

FIG. 3 illustrates a mobile dispensing station 300. The embodiment uses a number of canisters that contain a particular blend of the iodine-enriched water (with minerals and/or vitamins if these are used). Iodine canisters 340, 341, 342 are provided and loaded or stored into the multifunctional portable spray station 300. The iodine solution in canister 341 feeds a chilling system 339 to reduce the temperature of the iodine solution. The chilling system 339 feeds iodine solution to an ultrasonic sonic humidifier 375 and/or impeller humidifier 376. The iodine enriched humidified air from one of the humidifiers 375 or 376 is then directed through air columns 325. Air columns 325 have adjustable vents 326 at a terminal end thereof. Flexible, extendable air tubes 310 can be attached to the vents 326 and iodine enriched humidified air is delivered to adjustable air outlets 301 via the vents 326 and tubes 310. The air tubes 310 can be telescoping to allow for more flexibility in the placement of the outlets 301.

The iodine solution in canister 340 feeds the ultrasonic humidifier 3775. Vitamins and minerals in canisters 350 also feed the ultrasonic humidifier 377. The humidifier 377 is designed to feed a nebulizer column 305, which can be used by an individual needing therapy using the iodine enriched humidified air, or the air with the desired vitamins and/or minerals.

The iodine solution in canister 342 is designed to feed the trigger spray apparatus 318 housed in handle 319. The trigger spray apparatus is intended to be used to apply the iodine-enriched solution from the canister 342 for surface application, for example.

The station 300 is equipped with a roller wheel 320, which stores an extendable hose (not shown) that is interconnected between apparatus 318 and the iodine canister 342. The hose allows the spray apparatus 318 to extend from the station 300 for application of the iodine-enriched water in canister 342 for application to a particular area.

The station 300 also includes a programmable controller 330 that is a failsafe controller that controls all aspects of the system including but not limited to water particle delivery size, molecular retention control through thermodynamics and iodine concentration levels being delivered.

In use of the station 300, distinct iodine solution canisters are installed in the station to work with the different functions that are provided by the mobile station. One of the iodine canisters on the lower level feeds the two humidifiers by way of a cooling system. The cool humidified iodine enriched air is directed up the air columns and then distributed through the extendable, flexible air vents into a room or establishment.

The other two iodine canisters can be located on the second level of the device. One iodine canister on the second level feeds a nebulizer humidifier that produces fine water particle sizes enriched with iodine and or minerals and vitamins. A face mask as part of the nebulizer allows for direct inhalation of the iodine enriched air for human health applications.

The second iodine canister on the second level feeds a coarse spray trigger system. The hand held device is removed from it holder and is used to spray iodine either as hard surface disinfectant or as a topical disinfectant spray for healing wounds.

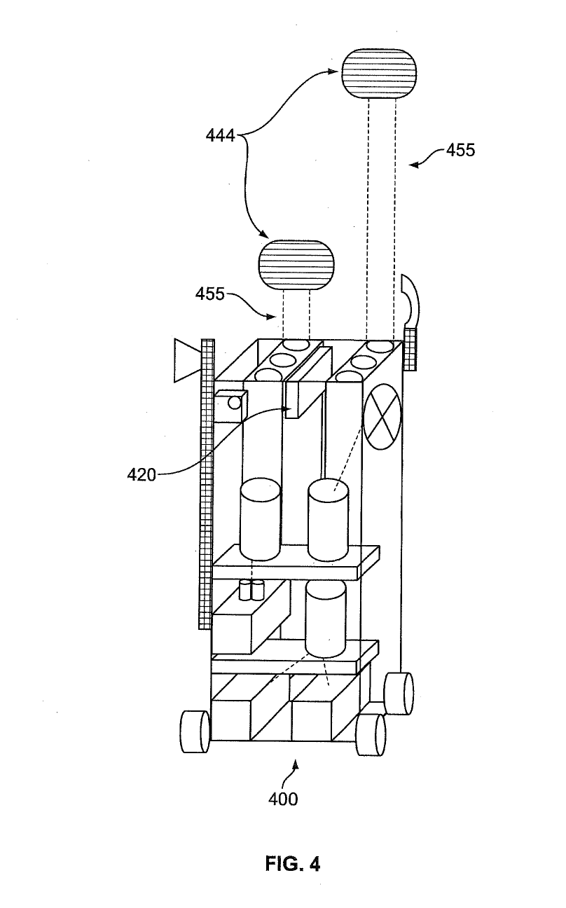

FIG. 4 shows another embodiment of a mobile station and is designated by the reference numeral 400. In this embodiment, an oscillation is included in the station to better distribute the iodine-enriched solution to a designed area.

The station 400 is identical in many respects when compared to the station 300 of FIG. 3. The main difference is the addition oscillating motor system 420. The extendable vent tubes 455 are intended to be rigid as are vent outlets 444. Vent tubes 455 are connected to oscillating motor system 420.

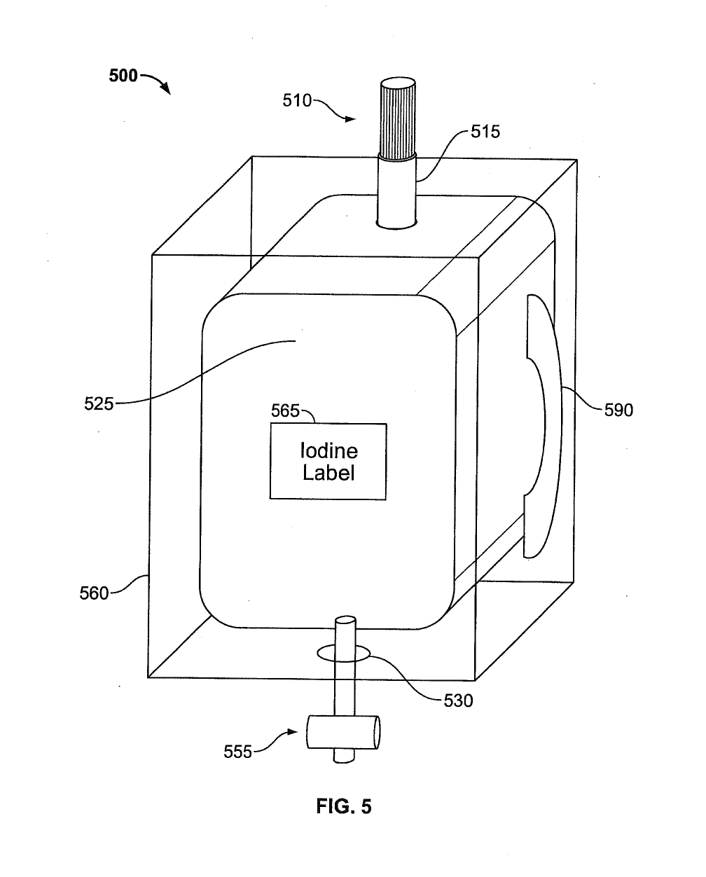

FIG. 5 illustrates a container for holding of the iodine solution for use in the FIGS. 3 and 4 apparatus and the container is designated by the reference numeral 500. The solid container 500 includes a container body 525, which can be fabricated from either plastic or stainless steel and can include a carry handle 590 to easy transport of the container body 525. The container body 525 can be a collapsible chemical inert plastic that retains the iodine solution. The iodine solution to be stored in the container body 525 is filled through an inlet tube 515. A concentration specific mated fitting 510 connects to its counter part on the iodine filling station represented in FIGS. 1 and 2.

An iodine dispensing tube 530 has a standard roller drip mechanism that feeds iodine to the humidification systems represented in FIGS. 3 and 4. Of course, other mechanisms could be used to allow the iodine enriched water in the container body 525 to get to the desired location in the station, e.g., one of the humidification systems, the trigger spray, and nebulizer.

FIG. 5 also shows the label 565, which would identify the contents of the body 525, e.g., iodine concentration, mineral concentration, vitamin concentration, the type of fitting that is used to fill the body 525, etc. The body 525 could be enclosed in another and rigid shell 560, if so desired.

Through use of the invention, the iodine enriched humidified air is directionally controlled through telescopic tubing vents emanating from the mobile chemical delivery apparatus or station. A coarse surface spray iodine disinfectant solution for topical wound disinfection and surface disinfection is delivered by way of an extendable hose and hand held spray trigger device. A nebulizer attachment on the mobile chemical delivery station provides for iodine, vitamin, mineral, and vaccine inhalation therapy.

The apparatus contains aqueous iodine disinfectant solutions retained within canisters with iodine concentration specific mated intake and return fittings. The iodine canisters within the chemical delivery system proportionally feed the misting systems and the coarse spray system.

A method and apparatus for producing and delivering formulated aqueous iodine solutions under static and dynamic flow conditions; retaining, modifying and dispending said aqueous iodine solutions. The iodine dispensing tanks have concentration specific mated dispensing fittings.

The mobile chemical delivery station provides an aqueous iodine surface and aerosol disinfectant for use in but not limited to operating rooms, post operating rooms, intensive care, trauma centers, clinics, and other areas where surface disinfection and air quality pose a health risk. Other uses of the surface and aerosol iodine disinfectant for example are cruise ships, airplanes, trains, office buildings, and apartment buildings, food processing facilities, restaurants, schools, nursing homes and the residential markets. The inventive method provides a plurality of iodine solutions in a mobile, portable aqueous iodine dispensing system comprising:

a) a self contained aqueous iodine concentrate generator;

b) a water filter and a tank to retain and dispense a high quality water supply to be used as the blend water for the iodine concentrate;

c) a primary iodine concentration retention and dispensing tank with a collapsible chemical inert sealed bladder;

d) secondary diluted iodine concentration and dispensing tanks with collapsible chemical inert sealed bladders

e) a system to control ph;

f) a vitamin and mineral dispensing system; and

f) a controller to operate the system

The method and device that operationally interacts with the system as described above has a plurality of spray/misting apparatuses to deliver either pure aqueous iodine solutions or a plurality of aqueous iodine mixtures with both vitamins and minerals and or vaccines to be delivered by a portable mobile variable spray station. The station includes:
a) variable spray delivery systems to disinfect surfaces including both aerosol systems and a trigger pump spray apparatus;
b) variable spray delivery systems to disinfect air;
c) a variable spray delivery system for inhalation therapy
d) a several refillable aqueous iodine canisters;
e) a plurality of soluble mineral, vitamin and or vaccine canisters; and
f) distinct mated iodine concentration and label specific connector fittings.

The station can include a plurality of flexible telescopic air duct delivery apparatuses and a cooling system to provide a method to thermodynamically control the aqueous iodine solutions feeding the humidification systems.

The refillable aqueous iodine canisters can have has concentration and label specific fittings that are distinctly mated for filling. The refillable aqueous iodine canisters can have an interior chemical inert bladder that is sealed and collapsible, and the bladder can have a drip tube to feed the aqueous solutions to the humidifiers. The station produces a plurality of variations of iodine, and or iodine and mineral and vitamin enriched air via a nebulizer, ultrasonic humidification and impeller humidification apparatuses. The nebulizer can provide vaccines through inhalation as well as variations of iodine, and or iodine and mineral and vitamin enriched air through inhalation. The programmable controller has built in safety features to operate the system.

As such, an invention has been disclosed in terms of preferred embodiments thereof which fulfills each and every one of the objects of the present invention as set forth above and provides a new and improved system and method to delivery iodine-enriched water with or without vitamins, minerals, vaccines to a desired location or individual.

Of course, various changes, modifications and alterations from the teachings of the present invention may be contemplated by those skilled in the art without departing from the intended spirit and scope thereof. It is intended that the present invention only be limited by the terms of the appended claims.

1. An iodine enriched water delivery system comprising:
a) a plurality of fittings, each fitting configured differently from the other fittings and adapted to connect to a container having iodine enriched water and having a matching mating fitting, the different fittings allowing for connection of a container having a defined iodine concentration solution; and one or a combination of the following systems:
b) a humidifier system having one or both of an ultrasonic and an impeller humidifier, the humidifier system having:
i) one of the mating fittings adapted to connect to a container containing a solution of defined iodine concentration for humidification;
ii) a chiller adapted to receive the solution to reduce a temperature thereof and form a cooled solution and deliver the cooled solution to one or both humidifiers; and
iii) one or more vent tubes for delivery of the cooled solution to atmosphere;
c) a nebulizer system having a mask to dispense a nebulized solution via the mask to a user, the nebulizer system having one of the mating fittings to connect to a container of defined iodine concentration for nebulization;
d) a trigger spray system having a trigger spray to spray the iodine enriched water on a surface, the trigger spray system having one of the mating fittings to connect to a container of defined iodine concentration for surface spraying.

2. The system of claim 1, wherein the humidifier system includes means to oscillate the one or more vent tubes.

3. The system of claim 1, wherein the one or more vent tubes can telescope to change a length thereof.

4. The system of claim 1, wherein the one or more vent tubes are either flexible or rigid.

5. The system of claim 1, further comprising a frame that can be rolled to move the following systems to different locations.

6. The system of claim 1, further comprising a controller that can control one or more of:
a) droplet size for humidification;
b) dispensing rate for humidification;
c) dispensing rate for nebulization;
d) droplet size for nebulization; and
e) temperature of the cooled solution exiting the chiller.

7. The system of claim 1, further including space to hold a plurality of the containers.

8. An iodine enriched water system comprising:
a) a filter adapted to connect to a source of water;
b) an iodine concentrate generator using filtered water from the filter;
c) a concentrated iodine solution storage tank to receive the concentrated iodine solution for the generator;
d) a plurality of pumps for delivering a quantity of the concentrated iodine solution to a plurality of outlet fittings,
e) means to add the filtered water, and optionally vitamins and minerals to the concentrated iodine solution to produce the iodine enriched water, wherein each fitting to be connected to a portable container is uniquely configured to produce an iodine enriched water having a predetermined concentration of iodine; and
f) a controller for regulating:
the amount of concentrated iodine solution to fill the portable containers via the outlet fittings,
the amount of filtered water to fill the portable containers via the outlet fittings;
the amount of minerals and vitamins, if added, to the portable containers via the outlet fittings.

9. The system of claim 8, further comprising a pH control for the filtered water.

10. The system of claim 8, wherein the system is portable so that it can be moved, and preferably rolled.

11. The system of claim 8, further comprising a plurality of portable containers,
each of the portable containers having a mating inlet fitting adapted to connect to only one of the outlet fittings, the container outlet fitting permitting dispensing of the iodine enriched water in the portable container.

12. An iodine enriched water system comprising:
a) a filter adapted to connect to a source of water;
b) an iodine concentrate generator using filtered water from the filter;
c) a concentrated iodine solution storage tank to receive the concentrated iodine solution for the generator;
d) a mixing manifold having a plurality of spigots, the mixing manifold configured to receive the concentrated iodine solution and mix it with the filter water and optionally vitamins and minerals to produce a plurality of iodine enriched waters of different concentrations, each plurality of pumps for delivering a quantity of the concentrated iodine solution to a plurality of outlet fittings,
e) means to add the filtered water and, optionally vitamins and minerals, to the concentrated iodine solution to produce the iodine enriched water, wherein each fitting to be connected to a portable container is uniquely configured to produce an iodine enriched water having a predetermined concentration of iodine; and
f) a controller for regulating:
the amount of concentrated iodine solution to fill the portable containers via the outlet fittings,
the amount of filtered water to fill the portable containers via the outlet fittings; and
the amount of minerals and vitamins, if added to the portable containers via the outlet fittings.

13-15. (canceled)

16. A method of delivery an iodine enriched water comprising:
producing a plurality of iodine enriched waters of different iodine concentration from a concentrated iodine solution, the iodine enriched waters optionally containing one or more minerals and vitamins;
delivering the iodine enriched waters by one of more of the following steps:
i) cooling and then humidifying the iodine enriched water of one iodine concentration and dispensing the humidified iodine enriched water to atmosphere;
ii) nebulizing the iodine enriched water of a second iodine concentration for use by a person; and
iii) spraying the iodine enriched water of a third iodine concentration on a surface.

17. The method of claim 16, wherein an oscillation is applied to the humidified iodine enriched water dispensed to atmosphere.

18. The method of claim 16, wherein the delivering step is done from a portable platform so that the delivering step can be performed in more than one location.

19. A method of making an iodine enriched water comprising:
providing a concentrated iodine solution;
mixing the concentrated iodine solution with filtered water and optionally one or more minerals and vitamins to form a plurality of iodine enriched waters of different concentrations; and
dispensing one or more of the plurality of iodine enriched waters for use or storage.

20. The method of claim 19, wherein each of the plurality of iodine enriched waters is stored in a storage container.

21. The method of claim 19, wherein the dispensing step uses a plurality of dispensing fittings, each dispensing fitting matched to an inlet fitting of one of the storage containers so that each storage container is adapted to hold an iodine enriched water of different iodine concentration.

22. The method of claim 16, wherein step (i) is used as the delivering step.

23. The system of claim 1, wherein the system of (b) is used.
